(19) United States
(12) Patent Application Publication
(10) Pub. No.: US 2024/0374152 A1
(43) Pub. Date: Nov. 14, 2024

(54) DISPLAY DEVICE

(71) Applicant: SAMSUNG DISPLAY CO., LTD., YONGIN-SI (KR)

(72) Inventors: CHUL KIM, Yongin-si (KR); GYEONG UB MOON, Yongin-si (KR); JONG YEOP AN, Yongin-si (KR); DONG WOOK YANG, Yongin-si (KR)

(21) Appl. No.: 18/516,440

(22) Filed: Nov. 21, 2023

(30) Foreign Application Priority Data
May 9, 2023 (KR) 10-2023-0060123

(51) Int. Cl. A61B 5/021; A61B 5/00

(52) U.S. Cl. CPC A61B 5/02141; A61B 5/681; A61B 5/6898; A61B 5/02427; A61B 2562/0247

(57) ABSTRACT

A display device includes a display panel including a plurality of light emitting elements, a cover glass covering an upper surface of the display panel and including a first area overlapping the display panel and a second area adjacent to the first area, a black matrix overlapping the second area of the cover glass, a pressure sensor overlapping the black matrix under the cover glass and configured to sense a pressure applied through the cover glass, and a set frame covering a lower surface of the display panel. The set frame includes a support contacting the pressure sensor on an upper surface of the set frame facing the cover glass.

### CROSS-REFERENCE TO RELATED APPLICATIONS

This application claims priority under 35 U.S.C. § 119 to Korean Patent Application No. 10-2023-0060123, filed on, May 9, 2023, the disclosure of which is incorporated by reference herein in its entirety.

### TECHNICAL FIELD

Embodiments of the present disclosure relate to a display device, and more particularly, to a display device including a pressure sensor.

### DISCUSSION OF RELATED ART

Multimedia electronic devices such as a television, a mobile phone, a tablet computer, a navigation device, a game console, etc., may include a display device that displays an image. The display device may be provided with various functions in addition to the function of displaying an image.

Recently, as interest in health management increases, interest and demand for a health care device capable of checking and managing one's health anytime, anywhere is increasing. For example, a portable blood pressure measurement device alone utilizes an independent light source, sensor, and display, and is inconvenient to carry separately in addition to a portable smartphone or tablet PC. Accordingly, a technology for monitoring a health state by including various sensors in the display device, such as a smartwatch is being developed.

### SUMMARY

Embodiments of the present disclosure provide a display device capable of measuring a pressure with increased reliability. For example, the display device may more accurately measure a blood pressure by securing a characteristic of a pressure sensor disposed under glass. For example, the display device may include a pressure sensor disposed to overlap an edge area under a cover glass to sense a pressure and a set frame covering a lower surface of the display panel. The set frame may include a support that contacts the pressure sensor on an upper surface of the set frame facing the cover glass. When a user touches the display device or the cover glass, a corresponding pressure may be concentrated (or amplified) to the pressure sensor by the support, and thus, the characteristic of the pressure sensor may be secured, and the blood pressure may be more accurately measured.

According to an embodiment of the present disclosure, a display device includes a display panel including a plurality of light emitting elements, a cover glass covering an upper surface of the display panel and including a first area overlapping the display panel and a second area adjacent to the first area, a black matrix overlapping the second area of the cover glass, a pressure sensor overlapping the black matrix under the cover glass and configured to sense a pressure applied through the cover glass, and a set frame covering a lower surface of the display panel. The set frame includes a support contacting the pressure sensor on an upper surface of the set frame facing the cover glass.

The pressure sensor may be disposed between the cover glass and the set frame.

The support may include a concavo-convex structure on one surface contacting the pressure sensor.

The pressure sensor may have a surface facing the support, the support may have a surface facing the pressure sensor, and an area of the surface of the support may be less than or equal to an area of the surface of the pressure sensor.

The pressure sensor may include one or more pressure sensor units each generating a pressure sensing signal.

The support may include one or more sub-supports respectively contacting the one or more pressure sensor units on the upper surface of the set frame.

The number of the one or more pressure sensor units and the number of the one or more sub-supports may be the same.

The display device may further include a driver configured to calculate pressure position information using the one or more pressure sensor units.

When a pressure is applied to the cover glass, pressure sensor units adjacent to a point where the pressure is applied among the one or more pressure sensor units may be used to calculate the pressure position information.

The driver may calculate the pressure position information based on a weighted average of pressure sensing signals generated from the pressure sensor units adjacent to the point where the pressure is applied.

The display panel may further include a light sensor configured to sense reflected light emitted from the plurality of light emitting elements and reflected from a target object.

The display device may further include a driver configured to calculate blood pressure information based on the pressure sensing signal and a light sensing signal generated by the light sensor.

According to an embodiment of the present disclosure, a display device includes a display panel including a plurality of light emitting elements, a cover glass covering an upper surface of the display panel and including a first area overlapping the display panel and a second area adjacent to the first area, a black matrix overlapping the second area of the cover glass, a pressure sensor overlapping an edge area of the first area under the display panel and configured to sense a pressure applied through the cover glass, and a set frame covering a lower surface of the display panel. The set frame includes a support contacting the pressure sensor on an upper surface of the set frame facing the display panel.

The pressure sensor may be disposed between the display panel and the set frame in the edge area, and the display panel may be disposed between the cover glass and the pressure sensor.

The edge area overlapping the pressure sensor may be an area adjacent to the second area.

The support may include a concavo-convex structure on one surface contacting the pressure sensor.

The pressure sensor may include one or more pressure sensor units each generating a pressure sensing signal.

The support may include one or more sub-supports respectively contacting the one or more pressure sensor units on the upper surface of the set frame.

### BRIEF DESCRIPTION OF THE DRAWINGS

The above and other features of the disclosure will become more apparent by describing in further detail embodiments thereof with reference to the accompanying drawings, in which:

FIG. 1 is a block diagram illustrating a display device according to an embodiment of the disclosure;

FIG. 2 is a plan view illustrating an embodiment of a disposition of a pressure sensor in the display device of FIG. 1;

FIG. 3 is a cross-sectional view taken along line I-I′ of FIG. 2;

FIG. 4 is a plan view illustrating an embodiment of the disposition of the pressure sensor in the display device of FIG. 1;

FIG. 5 is a cross-sectional view taken along line II-II′ of FIG. 4;

FIGS. 6 and 7 are diagrams illustrating embodiments of a portion of a support disposed near the pressure sensor in FIG. 3;

FIGS. 8 and 9 are plan views illustrating embodiments of a disposition of a pressure sensor including one pressure sensor unit and the support in the display device of FIG. 1;

FIG. 10 is a plan view illustrating an embodiment of a disposition of a pressure sensor including two or more pressure sensor units and the support in the display device of FIG. 1;

FIG. 11 is a plan view illustrating an example of a disposition of the pressure sensor and the support in the display device according to an embodiment of the disclosure;

FIG. 12 is a block diagram illustrating an example of the pressure sensor included in the display device;

FIGS. 13 and 14 are diagrams illustrating an operation of a sensing cell included in the pressure sensor;

FIG. 15 is a diagram illustrating an operation of calculating pressure position information in the display device; and

FIG. 16 is a cross-sectional view illustrating an example of a light sensor included in the display device.

### DETAILED DESCRIPTION OF THE EMBODIMENTS

Embodiments of the present disclosure will be described more fully hereinafter with reference to the accompanying drawings. Like reference numerals may refer to like elements throughout the accompanying drawings.

It will be understood that when a component such as a film, a region, a layer, etc., is referred to as being “on”, “connected to”, “coupled to”, or “adjacent to” another component, it can be directly on, connected, coupled, or adjacent to the other component, or intervening components may be present. It will also be understood that when a component is referred to as being “between” two components, it can be the only component between the two components, or one or more intervening components may also be present. It will also be understood that when a component is referred to as “covering” another component, it can be the only component covering the other component, or one or more intervening components may also be covering the other component. Other words used to describe the relationships between components should be interpreted in a like fashion.

Throughout the specification, when the term “includes” is used, it means that the portion may further include another component without excluding another component unless otherwise stated. “At least any one of X, Y, and Z” may be interpreted as one X, one Y, one Z, or any combination of two or more of X, Y, and Z (for example, XYZ, XYY, YZ, and ZZ). Here, “and/or” includes all combinations of one or more of corresponding configurations.

It will be understood that the terms “first,” “second,” “third,” etc. are used herein to distinguish one element from another, and the elements are not limited by these terms. Thus, a “first” element in an embodiment may be described as a “second” element in another embodiment.

It should be understood that descriptions of features or aspects within each embodiment should typically be considered as available for other similar features or aspects in other embodiments, unless the context clearly indicates otherwise.

As used herein, the singular forms “a”, “an” and “the” are intended to include the plural forms as well, unless the context clearly indicates otherwise.

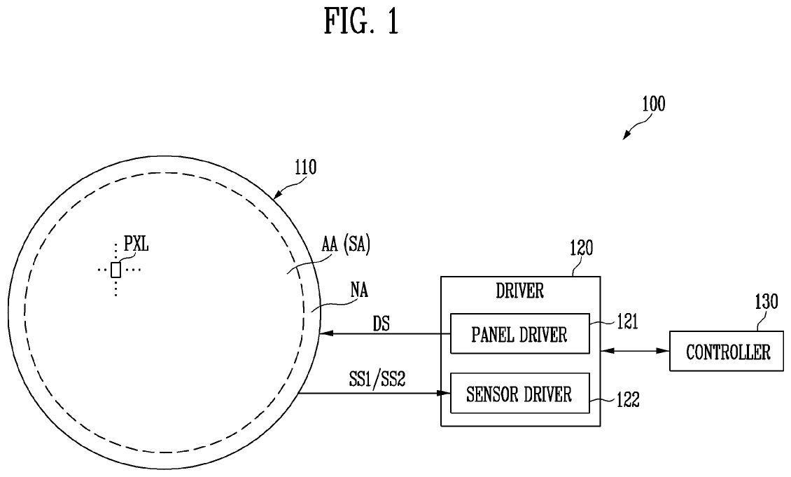

FIG. 1 is a block diagram illustrating a display device according to an embodiment of the disclosure. Referring to FIG. 1, the display device 100 may include a display panel 110, a driver 120, and a controller 130.

In an embodiment, the display panel 110 may include a plurality of light emitting elements. For example, the display panel 110 may include a display area AA in which an image is displayed and a non-display area NA in which an image is not displayed.

The display area AA may be an area where a plurality of pixels PXL (or a sub-pixel) is provided, and may be an active area. Here, each of the pixels PXL may include at least one light emitting element. For example, the display device 100 may display an image in the display area AA of the display panel 110 by driving the pixels PXL in response to image data input from outside of the display device 100.

In addition, the display area AA may include a sensing area SA (or a first area). The sensing area SA may include at least some pixels PXL among the pixels PXL provided in the display area AA.

As shown in FIG. 1, at least a portion of the display area AA may be set as a sensing area SA capable of sensing a touch, or the entire display area AA may be set as the sensing area SA. However, when the entire the display area AA is set as the sensing area SA, a blood pressure measurement operation may be performed only in a portion substantially touched by a user.

The non-display area NA may an area disposed around the display area AA and may be referred to as a non-active area. For example, the non-display area NA may include a line area, a pad area, various dummy areas, and the like.

As another example, the display panel 110 may include organic light emitting elements (e.g., organic light emitting diodes), inorganic light emitting elements (e.g., inorganic light emitting diodes), quantum dot/well light emitting elements (e.g., quantum dot/well light emitting diodes), and the like as the pixels PXL. Alternatively, the display panel 110 may be implemented as a liquid crystal display panel. In this case, the display device 100 may additionally include a light source such as a back-light unit.

In an embodiment, the display device 100 may include a sensor provided in the sensing area SA of the display panel 110. The sensor may be, for example, a blood pressure sensor including a pressure sensor that senses a pressure and a light sensor that senses reflected light. For example, the blood pressure sensor may be configured of the pressure sensor that senses a pressure applied to at least a portion of the display panel 110 and the light sensor that senses the reflected light incident through the display panel 110.

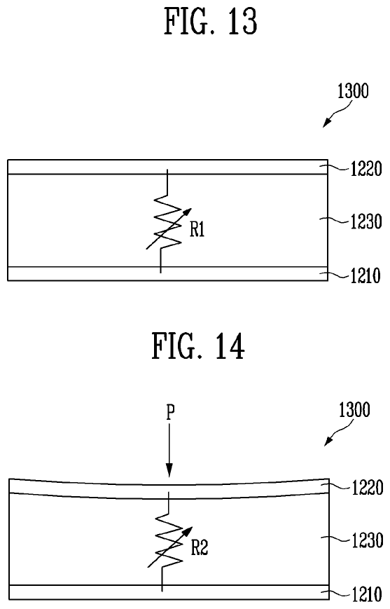

When a pressure is applied to the sensing area SA by a portion of a user's body (for example, a finger), the pressure sensor may sense an area (or the area) to which the pressure is applied and a magnitude of the pressure (for example, a pressure value or force) to output a corresponding electrical signal (for example, a voltage signal). A pressure sensing signal SS1 having different electrical characteristics according to the pressure may be output. The pressure sensor may be implemented as, for example, a general strain gauge type of pressure sensor or a capacitance type of pressure sensor. The pressure sensor is described in further detail below with reference to FIGS. 13 to 15.

When light emitted from a light source provided in the light sensor or a light source (for example, the pixels PXL) provided in the display panel 110 is reflected by a portion of the user's body, the light sensor may sense the reflected light and output a corresponding electrical signal (for example, a voltage signal) as a light sensing signal SS2. The light sensing signal SS2 having different electrical characteristics may be output according to a blood flow rate flowing through a blood vessel in a portion of the user's body. The light sensor may be implemented as a general photoplethysmography sensor (for example, a PPG sensor). Here, when the light sensor uses the pixels PXL provided in the display panel 110 as a light source without a separate external light source, a volume of the light sensor and the blood pressure sensor including the same may be reduced, and a manufacturing cost may be reduced. The light sensor are described in further detail below with reference to FIG. 16.

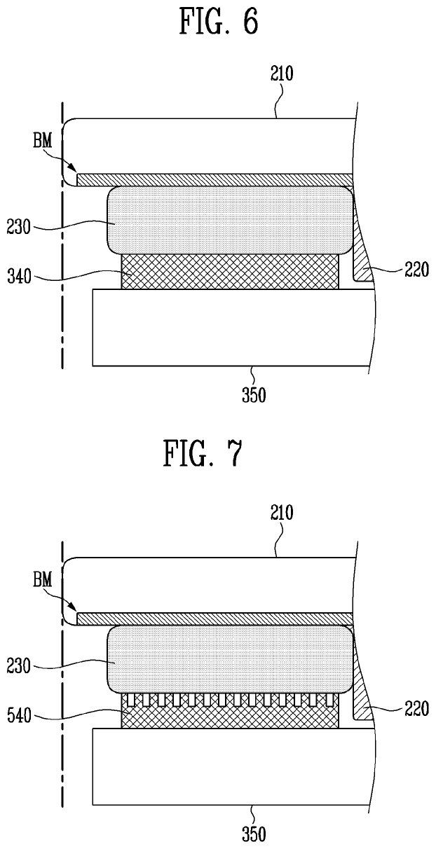

In an embodiment, the display device 100 may include a set frame that covers a lower surface of the display panel 110, and the set frame may further include a support (also referred to as a support member) that supports the pressure sensor. The support may concentrate the pressure applied through a cover glass to the pressure sensor without distributing the pressure. Accordingly, sensitivity of the pressure sensor disposed under the cover glass may be improved. The support is described in further detail below with reference to FIGS. 6 to 11.

In an embodiment, the driver 120 may include a panel driver 121 and a sensor driver 122. The panel driver 121 may drive the display panel 110 and the sensor driver 122 may measure information using a sensor.

The panel driver 121 may generate a signal for supplying a voltage to the display panel 110. For example, the panel driver 121 may include a driving circuit such as a data driving circuit configured to output a data voltage, a scan driving circuit configured to supply a scan signal, and an emission driver circuit configured to supply an emission signal.

The sensor driver 122 may receive a sensing signal from the sensor. For example, the sensor driver 122 may convert the sensing signal received from the sensor into a corresponding digital value and output the converted digital value. The sensor driver 122 may include a sensing circuit configured as an analog-to-digital converter that converts an analog voltage value into a digital value corresponding thereto when appropriate.

For example, the panel driver 121 may output a data signal DS corresponding to image data to the display panel 110. In addition, the sensor driver 122 may receive electrical signals (for example, the first sensing signal SS1 provided from the pressure sensor and the second sensing signal SS2 provided from the light sensor) from the pressure sensor. The sensor driver 122 may measure a user's blood pressure by calculating a change in a blood flow rate according to the pressure using the electrical signals.

Each of the panel driver 121 and the sensor driver 122 may be implemented as an integrated circuit and mounted on a flexible circuit board. The panel driver 121 may be connected to the display panel 110 through the flexible circuit board, and the sensor driver 122 may be connected to the pressure sensor through the flexible circuit board. FIG. 1 shows the panel driver 121 and the sensor driver 122 separated from each other, but embodiments are not limited thereto. For example, the sensor driver 122 and the panel driver 121 may be mounted in one integrated chip (IC) or may be configured as separate ICs according to embodiments.

In an embodiment, the display device 100 may include the controller 130 that controls an operation timing of the driver 120. The controller 130 may generate and output a control signal that controls the operation timing of the driver 120 and the image data. In addition, the driver 120 may receive the control signal from the controller 130 to control a timing (or a length of a period) of sensing (for example, reading out) using the light sensor and the pressure sensor.

Although a smartwatch is described as an example of the display device 100, the display device 100 may be applied to another type of electronic device such as, for example, a computer, a laptop, a cellular phone, a smartphone, a personal digital assistant (PDA), a portable multimedia player (PMP), a digital TV, a digital camera, a portable game console, a navigation device, a wearable device, an Internet of Things (IoT) device, an Internet of Everything (IoE) device, an e-book, a virtual reality (VR) device, an augmented reality (AR) device, a vehicle navigation device, a video phone, a surveillance system, an auto focus system, a tracking system, a motion sensing systems, or the like.

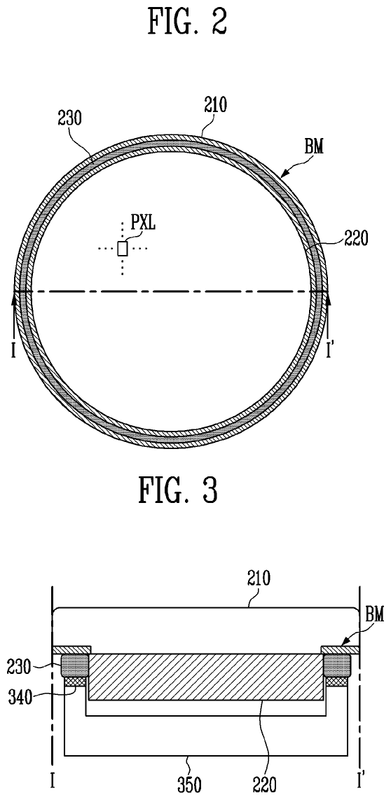

FIG. 2 is a plan view illustrating an embodiment of a disposition of the pressure sensor in the display device of FIG. 1. FIG. 2 is an enlarged plan view of a portion of the display device 100 of FIG. 1. FIG. 2 shows a cover glass 210 and a display panel 220 to describe a position where the pressure sensor 230 is disposed. The display device 100 may include the cover glass 210, the display panel 220, and a pressure sensor 230. In addition, in an embodiment, a set frame including a support that supports the pressure sensor 230 may be further included.

In an embodiment, the cover glass 210 may include a first area covering an upper surface of the display panel 220 and overlapping the display panel 220, and a second area adjacent to the first area (e.g., surrounding the first area). The cover glass 210 may be a protective member disposed at the uppermost end that covers all surfaces of the display panel 220, and may absorb shock applied from outside of the display device 100 to prevent the display panel 220 positioned thereunder from being damaged. For example, the cover glass 210 may be a substantially transparent light-transmitting substrate and may have a multilayer structure selected from a glass substrate, a plastic film, and a plastic substrate. In addition, the cover glass 210 may further include, for example, a polarizing plate, an antireflection layer, or a touch sensor layer (touch electrode layer) disposed between the cover glass 210 and the display panel 220.

However, similar to a smartwatch, in a case where the pressure sensor 230 is disposed under the cover glass 210 and the display panel 220, when the cover glass 210 has a predetermined thickness or more, a pressure applied to the cover glass 210 may not be adequately transferred to the pressure sensor 230. Here, the predetermined thickness may be about 1 to 2T (mm), but may change according to the hardness of the cover glass 210. Accordingly, the position at which the pressure sensor 230 is disposed may have a bearing on securing a desired characteristic of the pressure sensor. For example, a degree at which the cover glass 210 having the predetermined thickness or more is bent may be relatively greater in a case where a pressure is applied to an edge compared to a case where a pressure is applied to a center. Therefore, when the pressure sensor 230 is disposed under a black matrix BM of the cover glass 210 adjacent to an edge area of the display panel 220, a desired characteristic of the pressure sensor 230 may be secured.

The black matrix BM may overlap the second area of the cover glass 210. In other words, an area of the display device 100 overlapping the black matrix BM may be defined as the second area. In an embodiment, the black matrix BM is formed of a photosensitive resin to which black pigment is added so as to overlap the second area which is an area from an edge to a point spaced apart from the edge by a predetermined distance along a circumference of the cover glass 210. Thus, an area where the black matrix BM is formed may become the non-display area NA, and an area where the black matrix BM is not formed may become the display area AA.

Referring to FIG. 2, the pressure sensor 230 may be disposed to overlap the black matrix BM at an area under the cover glass 210, and may sense an external pressure applied through the cover glass 210 (for example, a user's finger). Thus, in an embodiment, the pressure sensor 230 may be disposed along the circumference of the cover glass 210. In addition, the pressure sensor 230 may be disposed between the cover glass 210 and the set frame. For example, the pressure sensor 230 may be disposed on an upper surface of the set frame facing the cover glass 210 while overlapping the black matrix BM at the area under the cover glass 210. The pressure sensor 230 may be disposed parallel to the display panel 220 and may be disposed to contact the support of the set frame.

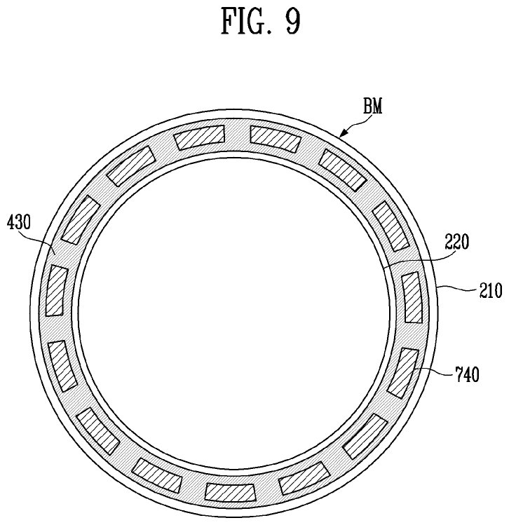

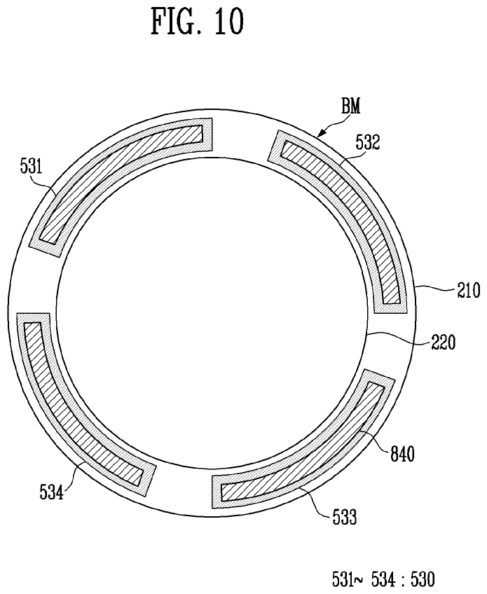

In addition, the pressure sensor 230 may include one or more pressure sensor units each generating a pressure sensing signal. However, when a plurality of pressure sensor units is included, the pressure sensor units may be disposed at a regular interval entirely under the black matrix BM of the cover glass 210, which may prevent error occurrence due to a position of an applied pressure. This is described in further detail below with reference to FIGS. 9 and 10.

FIG. 3 is a cross-sectional view taken along line I-I′ of FIG. 2.

Referring to FIG. 3, an example in which the pressure sensor 230 is disposed to overlap the black matrix BM is described. The pressure sensor 230 may be disposed to overlap the black matrix BM under the cover glass 210. In addition, the support 340 of the set frame 350 may be disposed to overlap the pressure sensor 230. That is, in the display device 100 according to an embodiment, the cover glass 210, the pressure sensor 230, and the support 340 of the set frame 350 may be sequentially disposed from the uppermost end. Accordingly, the support 340 may concentrate the pressure applied to the cover glass 210 to the pressure sensor 230 without distributing the pressure.

In an embodiment, the support 340 may be disposed to contact a lower surface of the pressure sensor 230. In addition, the pressure sensor 230 may have a first surface facing the support 340, the support 340 may have a second surface facing the pressure sensor 230, and the area of the second surface may be less than or about equal to that of the first surface. For example, a width (the area corresponding thereto) of a horizontal direction on the first surface facing the support 340 of the pressure sensor 230 may be less than a width (the area corresponding thereto) of the horizontal direction on the second surface facing the pressure sensor 230 of the support 340. Alternatively, the width of the horizontal direction on the first surface of the pressure sensor 230 may be about equal to the width of the horizontal direction on the second surface of the support 340. That is, since the area of the second surface of the support 340 is not greater than the area of the first surface of the pressure sensor 230, the pressure applied to the cover glass 210 may be concentrated to the pressure sensor 230 by the support 340.

The set frame 350 may cover a lower surface of the display panel 220. The set frame 350 may be disposed to cover the lower surface of the display panel 220 to support the cover glass 210, the display panel 220, and the pressure sensor 230. In addition, the set frame 350 may include the support 340 disposed to contact a lower surface of the pressure sensor 240 to support the pressure sensor 240. For example, the support 340 of the set frame 350 may protrude from an upper surface of the set frame 350 facing the cover glass 210 and may be disposed to contact the lower surface of the pressure sensor 230. Therefore, when the cover glass 210 is pressed, the pressure sensor 230 may accurately sense the pressure by the support 340.

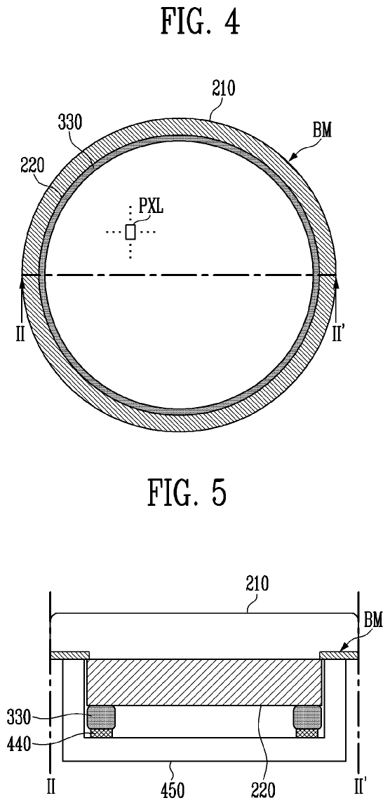

FIG. 4 is a plan view illustrating an embodiment of the disposition of the pressure sensor in the display device of FIG. 1.

FIG. 4 is a plan view illustrating an example of the display device 100 of FIG. 2. FIG. 4 shows the cover glass 210 and the display panel 220 to describe the position where the pressure sensor 330 is disposed. However, different from FIG. 2, in the display device 100 of FIG. 4, the pressure sensor 330 may be disposed under the display panel 220 instead of the cover glass 210.

In an embodiment, the cover glass 210 may include a first area covering an upper surface of the display panel 220 and overlapping the display panel 220, and a second area surrounding the first area. In addition, the pressure sensor 330 may be disposed to overlap an edge area of the first area under the display panel 220 to sense the pressure applied through the cover glass 210.

Still referring to FIG. 4, the pressure sensor 330 may be disposed between the display panel 220 and the set frame at the edge area of the first area. For example, the pressure sensor 330 may be disposed in an area adjacent to the second area of the first area as the edge area of the first area overlapping the display panel 220. Thus, in an embodiment, the pressure sensor 330 does not overlap the black matrix formed along the circumference of the cover glass 210, and is disposed under the display panel 220 overlapping an edge area of the display panel 220 adjacent to the black matrix.

In addition, the pressure sensor 330 may be disposed to be attached to the lower surface of the display panel 220 through a separate adhesive layer. Alternatively, the pressure sensor 330 may be directly formed on the display panel 220 through a process performed subsequent to a process of manufacturing the display panel 220. For example, the pressure sensor 330 may be disposed at least partially (with at least one electrode layer) inside the display panel 220, and in this case, a first electrode or a second electrode may be disposed between electrodes of the display panel 220. Although an example in which the pressure sensor 330 is disposed on the lower surface of the display panel 220 is described, embodiments of the disclosure are not limited thereto.

FIG. 5 is a cross-sectional view taken along line II-II′ of FIG. 4.

Referring to FIG. 5, an example in which the pressure sensor 330 is disposed to overlap the edge area of the first area of the display panel 220 is described. The pressure sensor 330 may overlap the edge area of the first area of the display panel 220 adjacent to the black matrix BM. In addition, the pressure sensor 330 may be disposed between the display panel 220 and the set frame at the edge area of the first area, and the display panel 220 may be disposed between the cover glass 210 and the pressure sensor 330. That is, in the display device 100 according to an embodiment, the cover glass 210, the display panel 220, the pressure sensor 330, and the support 440 of the set frame 450 may be sequentially disposed from the uppermost end.

The support 440 of the set frame 450 may be disposed to overlap the pressure sensor 330. In addition, the support 440 may be disposed to contact a lower surface of the pressure sensor 330. Accordingly, the support 440 may concentrate the pressure applied to the cover glass 210 and the display panel 220 to the pressure sensor 330 without distributing the pressure.

In addition, the pressure sensor 230 may have a first surface facing the support 440, the support 440 may have a second surface facing the pressure sensor 330, and the area of the second surface may be less than or about equal to that of the first surface. For example, a width (the area corresponding thereto) of a horizontal direction on the first surface facing the support 440 of the pressure sensor 330 may be less than or about equal to a width (the area corresponding thereto) of the horizontal direction on the second surface facing the pressure sensor 330 of the support 440. That is, since the area of the second surface of the support 440 is not greater than the area of the first surface of the pressure sensor 330, the pressure applied to the cover glass 210 may be concentrated to the pressure sensor 330 by the support 440.

The set frame 450 may cover the lower surface of the display panel 220. In addition, the set frame 450 may be disposed on the lower surface of the cover glass 210 to support the cover glass 210, the display panel 220, and the pressure sensor 330. In addition, the set frame 450 may include the support 440 disposed to contact the lower surface of the pressure sensor 330 and support the pressure sensor 330. For example, the support 440 of the set frame 450 may protrude from an upper surface of the set frame 450 facing the cover glass 210 and may be disposed to contact the lower surface of the pressure sensor 330. Accordingly, when the cover glass 210 is pressed, the pressure sensor 330 may accurately sense the pressure by the support 440.

In addition, the pressure sensor 330 may include one or more pressure sensor units each generating a pressure sensing signal. However, when a plurality of pressure sensor units is included, the pressure sensor units may be disposed at a regular interval entirely under the edge area of the display panel 220, which may prevent error occurrence due to a position of an applied pressure.

FIGS. 6 and 7 are diagrams illustrating embodiments of a portion of the support disposed near the pressure sensor in FIG. 3.

FIGS. 6 and 7 are side views taken along the line I-I′ of FIG. 2. In FIG. 6, the set frame 350 may include the support 340 contacting the pressure sensor 230 on the upper surface of the set frame 350 facing the cover glass 210.

The support 340 may include sub-supports (also referred to as sub-support members) spaced apart from each other on one surface contacting the pressure sensor. The sub-supports are described in further detail below with reference to FIGS. 9 to 11.

Referring to FIG. 6, the support 340 may be formed of a material having a relatively large elasticity modulus. Alternatively, the support 340 may be formed of a material that is not deformed by a repeated pressure. For example, the support 340 may be formed of the same material extended from the set frame 350 as a metal material or a conductive material such as copper or aluminum. Alternatively, the support 340 may be formed of a polymer resin such as, for example, acryl resin or epoxy resin.

In an embodiment, the support 340 may be disposed to contact the lower surface of the pressure sensor 230 on the upper surface of the set frame 350 facing the cover glass 210. For example, the support 340 may be disposed to overlap sensing cells of the pressure sensor 230. Here, the first electrode and the second electrode included in the sensing cells may overlap each other on a plane. That is, the area of one surface of the support 340 facing the pressure sensor 230 may be smaller than or equal to the area of each of the sensing cells.

The support 340 is shown as having a side shape of a rectangle, but may also have a side shape of, for example, a trapezoid, a triangle, or the like. That is, the support 340 may have various side shapes, but is not limited thereto.

Referring to FIG. 7, a support 540 may include a concavo-convex structure on one surface contacting the pressure sensor 230. For example, the support 540 may include a concavo-convex structure of which a cross section of a protrusion and a depression has a triangular, quadrangular, polygonal, circular, elliptical, or irregular shape as a lattice shape in which the protrusion and the depression are repeated on the one surface contacting the pressure sensor 230. Since the support 540 includes the concavo-convex structure on one surface, the pressure may be concentrated by the protrusion and an air gap may be formed between the support 540 and the pressure sensor 230, which may increase accuracy of the pressure.

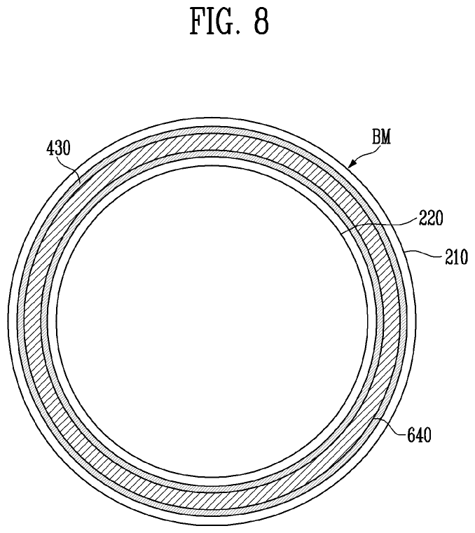

FIGS. 8 and 9 are plan views illustrating embodiments of a disposition of a pressure sensor including one pressure sensor unit and the support in the display device of FIG. 1.

FIGS. 8 and 9 are plan views illustrating the display device 100 of FIG. 2 as a reference. Referring to FIGS. 8 and 9, supports 640 and 740 contacting the pressure sensor 430 may include one or more sub-supports respectively contacting one or more pressure sensor units on the upper surface of the set frame. As a result, the supports 640 and 740 may concentrate the pressure applied to the cover glass 210 to the pressure sensor 430.

Referring to FIG. 8, when the pressure sensor 430 includes one pressure sensor unit, the support 640 may be integrally formed. In this case, the support 640 may include one sub-support contacting one pressure sensor unit on the upper surface of the set frame. The pressure applied to the cover glass 210 may be transferred to the pressure sensor 430 by the support 640 and evenly distributed to one surface contacting the pressure sensor 430. For example, when one surface of the support 640 that is in contact with the pressure sensor 430 is relatively large, the pressure may be unnecessarily distributed by the support 640. On the other hand, when the surface of the support 640 that is in contact with the pressure sensor 430 is relatively small, the pressure may not be unnecessarily distributed and may be concentrated to each of the sensing cells, which are a minimum unit of pressure sensing. Accordingly, in the integrated support 640 for one pressure sensor unit, the area of the second surface facing the pressure sensor 430 may be less than or about equal to the area of the first surface facing the support 640 of the pressure sensor 430. For example, the support 640 may include the concavo-convex structure on the second surface facing the pressure sensor 430.

Referring to FIG. 9, when the pressure sensor 430 includes one pressure sensor unit, a support 740 may be distributed to a plurality of bodies. In this case, the support 740 may include a plurality of sub-supports contacting one pressure sensor unit on the upper surface of the set frame. For example, the sub-supports may be spaced apart at a regular interval along the pressure sensor unit and arranged in a line. However, the sub-supports may also be arranged in two lines, but are not limited thereto. That is, the plurality of sub-supports may be disposed along the circumference of the cover glass 210 at a regular interval entirely, which may prevent error occurrence due to a point where the pressure is applied to the cover glass 210.

FIG. 10 is a plan view illustrating an embodiment of a disposition of a pressure sensor including two or more pressure sensor units and the support in the display device of FIG. 1.

Referring to FIG. 10, the pressure sensor 530 may generate a pressure sensing signal at a plurality of different points by two or more pressure sensor units 531 to 534. In addition, a support 840 may include two or more sub-supports respectively contacting one or more pressure sensor units on the upper surface of the set frame.

In an embodiment, the number of two or more pressure sensor units and the number of two or more sub-supports may be the same. When the pressure sensor 530 that senses the pressure at an area under the cover glass 210 includes N pressure sensor units, where N is a positive integer, the support 840 may include N sub-supports contacting each pressure sensor unit in a one-to-one correspondence. For example, as shown in FIG. 10, when the pressure sensor 530 includes first to fourth pressure sensor units 531 to 534, the support 840 may include four sub-support so as to contact the respective pressure sensor unit in a one-to-one correspondence. However, embodiments are not limited thereto. For example, in an embodiment, the support 840 may include eight sub-supports so that two or more contact each pressure sensor unit. The number of sub-supports may be variously set in consideration of the number and the contact area of the pressure sensor units. Also in this case, the plurality of sub-supports may be spaced apart from each other at a regular interval, which may prevent error occurrence due to the point where the pressure is applied to the cover glass 210.

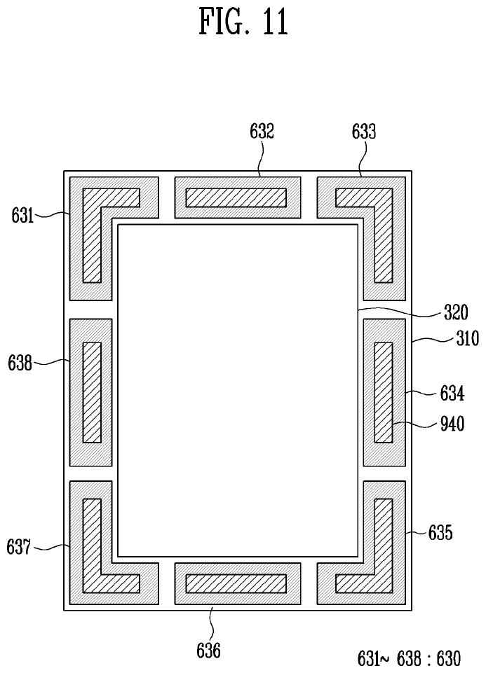

FIG. 11 is a plan view illustrating an example of a disposition of the pressure sensor and the support in the display device according to an embodiment of the disclosure.

As shown in FIG. 11, a cover glass 310 of the display device 100 may have a quadrangular shape on a plane. In addition, the cover glass 310 may have various shapes such as, for example, a circle, an ellipse, a triangle, and a quadrangle. However, as a pressure sensor 630 is disposed along a circumference of the cover glass 310, the pressure sensor 630 may be disposed in the same shape as that of the cover glass 310.

For example, when the cover glass 310 has a quadrangular shape, a black matrix overlapping an edge area of a first area overlapping the display panel 320 and a second area surrounding the first area may also have a quadrangular shape. Accordingly, the pressure sensor 630 may be disposed in a quadrangular shape to overlap the black matrix having the quadrangular shape. For example, when the pressure sensor 630 includes first to eighth pressure sensor units 631 to 638 as shown in FIG. 11, some pressure sensor units 631, 633, 635, and 637 among the first to eighth pressure sensor units 631 to 638 may be disposed at a corner of the quadrangular shape. In addition, a support 940 may include eight sub-supports contacting respective pressure sensor units in a one-to-one correspondence, and similar to the pressure sensor unit, some sub-supports among the sub-supports may be disposed at a corner of the quadrangular shape. However, embodiments of the disclosure are not limited thereto. For example, in an embodiment, a position at which the pressure sensor 630 is disposed and the number of pressure sensor units may be variously determined in an extent that a pressure applied to the cover glass 310 may be efficiently sensed.

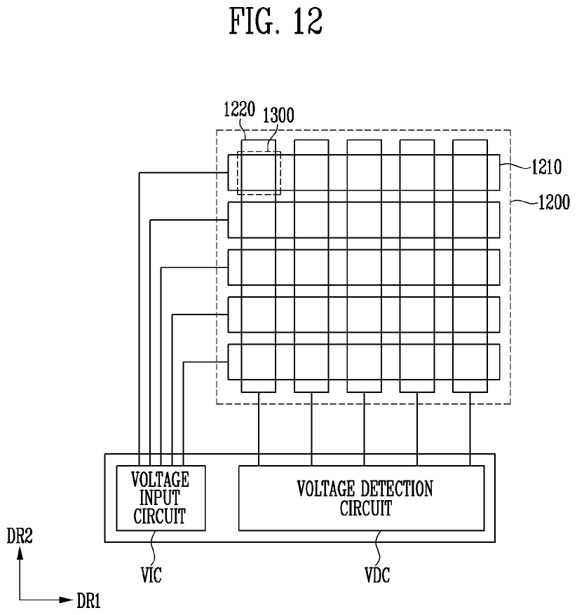

FIG. 12 is a block diagram illustrating an example of the pressure sensor included in the display device.

Referring to FIG. 12, the pressure sensor 230 may include a substrate 1200, first electrodes 1210, and second electrodes 1220. In addition, the pressure sensor 230 may include a voltage input circuit VIC and a voltage detection circuit VDC.

In an embodiment, the first electrodes 1210 may be disposed on the substrate 1200, may extend in a first direction DR1, and may be arranged spaced apart from each other along a second direction DR2. The second electrodes 1220 may extend in the second direction DR2 and may be arranged spaced apart from each other along the first direction DR1. The second electrodes 1220 partially overlap the first electrodes 1210, and the first electrodes 1210 and the second electrodes 1220 may be spaced apart from each other without directly contacting each other.

The first electrodes 1210 may be driving electrodes, and the second electrodes 1220 may be sensing electrodes. Sensing cells 1300 (nodes, or pressure sensing nodes) may be respectively formed in areas where the first electrodes 1210 and the second electrodes 1220 overlap. The sensing cells 1300 may independently sense a pressure of a corresponding location. Here, the sensing cell 1300 may be used as a pressure sensor unit included in the pressure sensor.

Lines for driving the sensing cells 1300 and receiving a sensing signal from the sensing cells 1300 may be connected. For example, the first electrodes 1210 may be electrically connected to the voltage input circuit VIC, and a specific voltage may be applied to the first electrodes 1210 from the voltage input circuit VIC. The second electrodes 1220 may be electrically connected to the voltage detection circuit VDC, and an electrical signal corresponding to the specific voltage may be output from the second electrode 1220 to the voltage detection circuit VDC.

In embodiments, any one of the pressure sensor 230 of FIG. 2, the pressure sensor 330 of FIG. 4, the pressure sensor 430 of FIG. 8, and the pressure sensor units 531 to 534 of FIG. 10, and any one of the pressure sensor units 631 to 638 of FIG. 11 may be implemented as the pressure sensor 230 of FIG. 12.

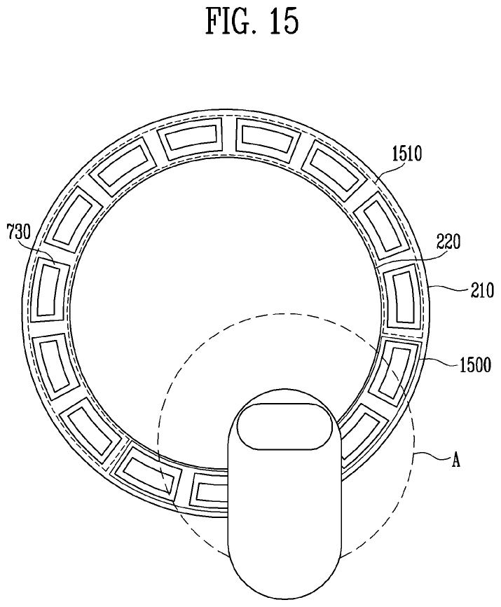

FIGS. 13 and 14 are diagrams illustrating an operation of the sensing cell included in the pressure sensor.

Referring to FIGS. 13 and 14, the pressure sensor 230 may include a plurality of sensing cells 1300, and each sensing cell 1300 may include a first electrode 1210, a second electrode 1220, and a variable resistance element 1230.

The first electrode 1210 may be formed of a conductive material or a transparent conductive material including a metal or an alloy thereof. For example, the metal may include gold (Au), silver (Ag), aluminum (Al), molybdenum (Mo), chromium (Cr), titanium (Ti), nickel (Ni), neodymium (Nd), copper (Cu), platinum (Pt), and the like. The transparent conductive material may include, for example, silver nanowire (AgNW), indium tin oxide (ITO), indium zinc oxide (IZO), antimony zinc oxide (AZO), indium tin zinc oxide (ITZO), zinc oxide (ZnO), tin oxide (SnO2), carbon nanotube, graphene, and the like.

The second electrode 1220 may be spaced apart from the first electrode 1210 and may include a conductive material. For example, the conductive material may be selected from a material capable of configuring the first electrode 1210, and may be formed of the same material as the material of the first electrode 1210 or a material different from that of the first electrode 1210.

The variable resistance element 1230 may be disposed between the first electrode 1210 and the second electrode 1220. The variable resistance element 1230 may be a component of which an electrical characteristic changes according to a degree of deformation. For example, the variable resistance element 1230 may include a material of which a resistance changes according to an external pressure between the first electrode 1210 and the second electrode 1220. For example, as force provided to the variable resistance element 1230 increases, the resistance of the variable resistance element 1230 may decrease, whereas as the force provided to the variable resistance element 1230 increases, the resistance of the variable resistance element 1230 may increase.

The variable resistance element 1230 may include a material of which the resistance changes according to a pressure. The variable resistance element 1230 may include a material referred to as a force sensitive material or a force sensitive resistor. For example, the variable resistance element 1230 may include at least one of piezo-electric materials such as lead zirconate titanate (PZT), barium titanate (BaTiO3), polytrifluoroethylene (PTrFE), and polyvinylidene fluoride (PVDF), a piezo-electric semiconductor such as poly crystal, PMN-PT single crystal, zinc oxide (ZnO), and molybdenum disulfide (MoS2), carbon powder, quantum tunneling composite (QTC), silicon, carbon nanotube, and graphene.

As another example, the variable resistance element 1230 may include a nanoparticle such as a nanotube, a nanocolumn, a nanorod, a nanopore, and a nanowire.

Alternatively, an elastic member may be provided between the first electrode 1210 and the second electrode 1220 instead of the variable resistance element 1230. As the first electrode 1210 and the second electrode 1220 are positioned spaced apart from each other, the first electrode 1210 and the second electrode 1220 may serve as a capacitor, and a capacitance may be formed between the first electrode 1210 and the second electrode 1220.

The elastic member may have an insulating property, which may prevent an electrical short circuit between the first electrode 1210 and the second electrode 1220. In addition, the elastic member may serve to alleviate an external impact, and to this end, the elastic member may have elasticity. For example, the elastic member may be deformed by an external pressure, and may have an elastic force capable of restoring an original state when the external pressure is removed.

The elastic member may be provided with a porous polymer to have elasticity. For example, the elastic members may include thermoplastic elastomer, polystyrene, polyolefin, polyurethane thermoplastic elastomers, polyamides, synthetic rubbers, polydimethylsiloxane, polybutadiene, polyisobutylene, polyurethanes, polychloroprene, polyethylene, silicone, and the like, and combinations thereof, but are not limited thereto.

Referring to FIG. 13 again, in a state in which a pressure is not applied to the sensing cell 1300 of the pressure sensor 230, the first electrode 1210 and the second electrode 1220 may be spaced apart by a first distance with the variable resistance element 1230 interposed therebetween. At this time, the variable resistance element 1230 may have a first resistance R1. Alternatively, a first capacitance may be formed between the first electrode 1210 and the second electrode 1220.

On the other hand, referring to FIG. 14, in a state in which the pressure is applied to the sensing cell 1300 of the pressure sensor 230, as the pressure is applied to the sensing cell 1300 of the pressure sensor 230 according to a user's touch or the like, a distance between the first electrode 1210 and the second electrode 1220 may change. Accordingly, as a shape of the variable resistance element 1230 changes, a resistance may change from the first resistance R1 to a second resistance R2. Therefore, a position, strength, and the like of the touch may be sensed with reference to a resistance change amount reflected in a pressure sensing signal output from the first electrode 1210 or the second electrode 1220.

Alternatively, when the pressure is applied to the sensing cell 1300 of the pressure sensor 230 according to the user's touch or the like, the distance between the first electrode 1210 and the second electrode 1220 may change. Accordingly, the capacitance of the first electrode 1210 and the second electrode 1220 may change from the first capacitance to a second capacitance. Accordingly, the position, the strength, and the like of the touch may be sensed with reference to a capacitance change amount reflected in the pressure sensing signal output from the first electrode 1210 and the second electrode 1220.

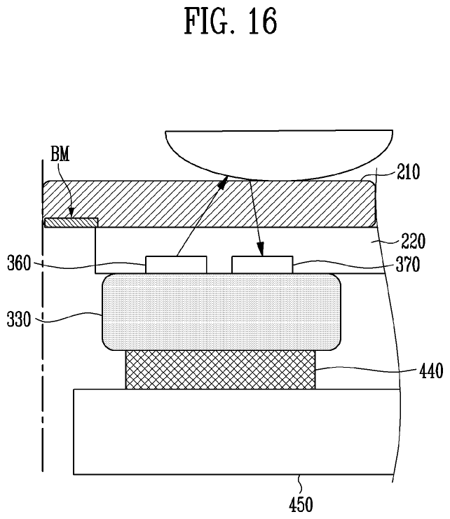

FIG. 15 is a diagram illustrating an operation of calculating pressure position information in the display device.

Referring to FIG. 15, embodiments of calculating pressure position information using one or more pressure sensor units included in the display device 100 are described. As described above, the display device 100 may include the driver 120 that calculates the press position information.

The pressure sensor 730 may include one or more pressure sensor units each generating the pressure sensing signal. The pressure sensor 730 may generate the pressure sensing signal at a plurality of different points by the respective pressure sensor units. In addition, the respective pressure sensor units may include respective channels and may be configured of a sensing cell, which is a minimum unit of pressure sensing. As described above, when the pressure sensor 730 includes the plurality of pressure sensor units, accuracy may be increased when sensing a pressure for the user's touch or the like, and multiple pressure sensing for multiple touches may be possible.

As shown in FIG. 15, the plurality of pressure sensor units may be included in one pressure sensor 730. For example, the pressure sensor 730 may be configured of 15 first electrodes and 15 second electrodes to include 15 pressure sensor units.

When the pressure is applied to the cover glass, the driver 120 may calculate the pressure position information by using pressure sensor units adjacent to a point where the pressure is applied among the one or more pressure sensor units.

Referring to FIG. 15, a degree at which the cover glass 210 is bent in a case where the pressure is applied to a point A of the cover glass 210 may be relatively greater than that of a case where the pressure is applied to a central portion. As the degree at which the cover glass 210 is bent increases, pressure sensor units adjacent to the point A may become active sensors, while pressure sensor units spaced apart by a predetermined distance from the point A may become non-active sensors. Here, the active sensor means a sensor that outputs a valid sensing signal, and the non-active sensor means a sensor that does not output a sensing signal or outputs an invalid sensing signal. Therefore, even though the same pressure is applied to the cover glass 210, the resistance change amount or the capacitance change amount reflected in the pressure sensing signal output to each pressure sensor unit may be different according to the point where the pressure is applied. In addition, since a wave for the pressure may occur by the cover glass 210 and the display panel 220, a pressure sensing signal different from an actual pressure sensing signal may be sensed.

Accordingly, the driver 120 may use only pressure sensor units positioned in a predetermined distance from the point A where the pressure is applied in pressure sensing. A magnitude of a signal sensed by pressure sensor units 1500 close to the point A may be greater than that of a signal sensed by pressure sensor units 1510 relatively distant from the point A. Accordingly, the driver 120 may further increase accuracy by using only some units having a large signal magnitude among the entire pressure sensor units and excluding units having a small signal magnitude.

The driver 120 may calculate the pressure position information based on a weighted average of the pressure sensing signals generated from the pressure sensor units adjacent to the point where the pressure is applied. For example, a weight allocated to the pressure sensing signal may be allocated as a larger value as the pressure sensor unit is adjacent to the point where the pressure is applied. The weight may be allocated as a value between 0 and 1 according to the magnitude of the pressure sensing signal generated from each of the pressure sensor units. Accordingly, the driver 120 may calculate the pressure position information by using a sum or an average of a value obtained by multiplying the pressure sensing signal output from each pressure sensor unit in correspondence with the magnitude of the pressure by the weight allocated according to a position where the pressure is applied. Accordingly, the driver 120 may prevent a calculation result of the pressure position information from being distorted because a specific pressure sensing signal has an excessively low or high intensity.

FIG. 16 is a cross-sectional view illustrating an example of the light sensor included in the display device.

Referring to FIG. 16, the display device 100 may include a light sensor that senses reflected light emitted from a plurality of light emitting elements and reflected from a target object. The light sensor may include a light emitting unit 360 and a light receiving unit 370 disposed spaced apart from each other.

The light emitting unit 360 may emit light and irradiate the target object (for example, a portion of a user's body, such as a finger) with the light. For example, the light emitting unit 360 may emit light from the pixels PXL in the sensing area SA (refer to FIG. 1) of the display panel 110. Alternatively, the light emitting unit 360 may emit light from at least one separate light source among, for example, a light emitting diode, an organic light emitting diode (OLED), an infrared emitting diode, and a laser diode included in the light sensor.

The light receiving unit 370 may sense the reflected light emitted from the light emitting unit 360 and reflected from the target object, and output an electrical signal corresponding to the reflected light. For example, the light receiving unit 370 may be implemented as a photodiode (organic photodiode).

In an embodiment, the light sensor may emit infrared light or green light through the light emitting unit 360. For example, when the light emitting unit 360 emits the infrared light, since a red blood cell (or hemoglobin (HB)) in a blood vessel absorbs the infrared light, an absorption amount for the infrared light may be measured through the light receiving unit 370. On the other hand, when the light emitting unit 360 emits the green light, since the red blood cell (or hemoglobin (HB)) absorbs the green light, an absorption amount for the green light may be measured through the light receiving unit 370. At this time, an absorption amount for emitted light by of the red blood cell (or hemoglobin (HB)) may change according to a volume change of a blood vessel.

The display device 100 may further include the driver 120 that calculates blood pressure information. The driver 120 may calculate the blood pressure information based on the pressure sensing signal SS1 generated from the pressure sensor and the light sensing signal SS2 generated from the light sensor. For example, the driver 120 may calculate the pressure based on force applied per area based on the pressure sensing signal SS1, calculate a blood flow rate based on the light sensing signal SS2, and calculate a blood pressure based on a change of the blood flow rate according to the pressure.

Referring to FIG. 16 again, while the user's finger presses the cover glass 210, the pressure on the sensing area SA may increase over time. As the pressure increases, the blood flow rate (or an amplitude of the blood flow rate) may decrease as blood gradually stops flowing in a corresponding portion of the finger.

For example, the driver 120 may calculate the blood flow rate (blood volume) based on an intensity of reflected light, reflectance, and light transmittance between the cover glass 210 and the display panel 220. At this time, the blood flow rate (blood volume) may appear in a form of oscillation in response to the volume change of the blood vessel. Accordingly, the driver 120 may calculate a change of the blood flow rate according to the pressure, and calculate the blood pressure information by applying a preset blood pressure inference algorithm to the change of the blood flow rate.

As described above, the display device 100 may more accurately measure the pressure through the pressure sensor 330 disposed at the edge of the cover glass 210 or the display panel 220 and the support 440 contacting the pressure sensor 330. Accordingly, the blood pressure information may be more accurately calculated.

According to embodiments of the disclosure, a display device capable of measuring a pressure with improved reliability is provided.

While the present disclosure has been particularly shown and described with reference to embodiments thereof, it will be understood by those of ordinary skill in the art that various changes in form and detail may be made therein without departing from the spirit and scope of the present disclosure as defined by the following claims.

What is claimed is:

1. A display device, comprising:
a display panel including a plurality of light emitting elements;
a cover glass covering an upper surface of the display panel and including a first area overlapping the display panel and a second area disposed adjacent to the first area;
a black matrix overlapping the second area of the cover glass;
a pressure sensor overlapping the black matrix under the cover glass and configured to sense a pressure applied through the cover glass; and
a set frame covering a lower surface of the display panel,
wherein the set frame includes a support contacting the pressure sensor on an upper surface of the set frame facing the cover glass.

2. The display device according to claim 1, wherein the pressure sensor is disposed between the cover glass and the set frame.

3. The display device according to claim 1, wherein the support includes a concavo-convex structure on one surface contacting the pressure sensor.

4. The display device according to claim 1, wherein the pressure sensor has a surface facing the support,
the support has a surface facing the pressure sensor, and
an area of the surface of the support is less than or equal to an area of the surface of the pressure sensor.

5. The display device according to claim 1, wherein the pressure sensor includes one or more pressure sensor units each generating a pressure sensing signal.

6. The display device according to claim 5, wherein the support includes one or more sub-supports respectively contacting the one or more pressure sensor units on the upper surface of the set frame.

7. The display device according to claim 6, wherein a number of the one or more pressure sensor units and a number of the one or more sub-supports are the same.

8. The display device according to claim 5, further comprising:
a driver configured to calculate pressure position information using the one or more pressure sensor units.

9. The display device according to claim 8, wherein when a pressure is applied to the cover glass, pressure sensor units adjacent to a point where the pressure is applied among the one or more pressure sensor units are used to calculate the pressure position information.

10. The display device according to claim 9, wherein the driver calculates the pressure position information based on a weighted average of pressure sensing signals generated from the pressure sensor units adjacent to the point where the pressure is applied.

11. The display device according to claim 5, wherein the display panel further includes a light sensor configured to sense reflected light emitted from the plurality of light emitting elements and reflected from a target object.

12. The display device according to claim 11, further comprising:
a driver configured to calculate blood pressure information based on the pressure sensing signal and a light sensing signal generated by the light sensor.

13. A display device, comprising:
a display panel including a plurality of light emitting elements;
a cover glass covering an upper surface of the display panel and including a first area overlapping the display panel and a second area adjacent to the first area;
a black matrix overlapping the second area of the cover glass;
a pressure sensor overlapping an edge area of the first area under the display panel and configured to sense a pressure applied through the cover glass; and
a set frame covering a lower surface of the display panel,
wherein the set frame includes a support contacting the pressure sensor on an upper surface of the set frame facing the display panel.

14. The display device according to claim 13, wherein the pressure sensor is disposed between the display panel and the set frame in the edge area, and
the display panel is disposed between the cover glass and the pressure sensor.

15. The display device according to claim 13, wherein the edge area overlapping the pressure sensor is an area adjacent to the second area.

16. The display device according to claim 13, wherein the support includes a concavo-convex structure on one surface contacting the pressure sensor.

17. The display device according to claim 13, wherein the pressure sensor includes one or more pressure sensor units each generating a pressure sensing signal.

18. The display device according to claim 17, wherein the support includes one or more sub-supports respectively contacting the one or more pressure sensor units on the upper surface of the set frame.
